(19) United States
(12) Patent Application Publication
(10) Pub. No.: US 2022/0257176 A1
(43) Pub. Date: Aug. 18, 2022

(54) NAP EVALUATION DEVICE, NAP EVALUATION SYSTEM, NAP EVALUATION METHOD, AND PROGRAM

(71) Applicant: Panasonic Intellectual Property Management Co., Ltd., Osaka (JP)

(72) Inventors: Yuki WAKI, Osaka (JP); Keita YOSHIMURA, Osaka (JP)

(21) Appl. No.: 17/625,610

(22) PCT Filed: Jun. 19, 2020
(86) PCT No.: PCT/JP2020/024170

§ 371 Date: Jan. 7, 2022

(30) Foreign Application Priority Data
Jul. 10, 2019 (JP) 2019-128764

(51) Int. Cl. A61B 5/00; A61B 5/11; G16H 50/30

(52) U.S. Cl. CPC A61B 5/4809; A61B 5/4812; A61B 5/4815; A61B 5/6891; A61B 5/486; A61B 5/11; G16H 50/30; A61B 5/72

(57) ABSTRACT

A first acquisition unit is configured to acquire time information regarding a time during which a user is napping. A second acquisition unit is configured to acquire a sleep stage of the user who is napping. A evaluation unit is configured to evaluate a quality of a nap in accordance with the time information and the sleep stage. The evaluation unit makes an evaluation that the lower a ratio of a second sleep time to a first sleep time is, the higher the quality of the nap. The first sleep time is a sleep time in light sleep during which the sleep stage is relatively light in a nap time from a start to an end of the nap. The second sleep time is a sleep time in deep sleep during which the sleep stage is relatively deep in the nap time.

### TECHNICAL FIELD

The present disclosure relates to nap evaluation devices, nap evaluation systems including the nap evaluation devices, nap evaluation methods, and programs. The present disclosure specifically relates to a nap evaluation device for evaluation of the quality of a nap, a nap evaluation system including the nap evaluation device, a nap evaluation method, and a program.

### BACKGROUND ART

Patent Literature 1 discloses a sleep monitoring device for evaluating the quality of sleep in accordance with sleep conditions in usual sleep. This sleep monitoring device calculates, from sleep physiology data during the sleep, a wakefulness time period, a REM sleep time period, a non-REM sleep time period, and a non-REM sleep depth as the sleep conditions. The sleep monitoring device obtains evaluation values for sleep evaluation items in accordance with the sleep conditions. Examples of the sleep evaluation items include a cycle in which REM sleep occurs, the total amount of deep non-REM sleep, and the number of times of wake-up after sleep onset. The sleep monitoring device evaluates the quality of the usual sleep in accordance with the evaluation values.

Incidentally, on the background of, for example, increased health management awareness of companies and the like, taking a nap of about 20 minutes during the day is supposed to help health promotion. When a person takes a short-time nap, he or she may want to evaluate the quality of the nap. However, the evaluation target of the sleep monitoring device described above is the usual sleep during which REM sleep occurs and deep non-REM sleep occurs. In the case of the nap taken during the day, short-time and light sleep during which no REM sleep occurs is preferable so that no sleep inertia occurs after the nap. The sleep monitoring device described above cannot evaluate the quality of the short-time nap.

### CITATION LIST

### Patent Literature

Patent Literature 1: JP 2007-319238 A

### SUMMARY OF INVENTION

It is an object of the present disclosure to provide a nap evaluation device configured to evaluate the quality of a nap, a nap evaluation system, a nap evaluation method, and a program.

A nap evaluation device of an aspect of the present disclosure includes a first acquisition unit, a second acquisition unit, and an evaluation unit. The first acquisition unit is configured to acquire time information regarding a time during which a user is napping. The second acquisition unit is configured to acquire a sleep stage of the user who is napping. The evaluation unit is configured to evaluate a quality of a nap in accordance with the time information and the sleep stage. The evaluation unit makes an evaluation that the lower a ratio of a second sleep time to a first sleep time is, the higher the quality of the nap. The first sleep time is a sleep time in light sleep during which the sleep stage is relatively light in a nap time from a start to an end of the nap. The second sleep time is a sleep time in deep sleep during which the sleep stage is relatively deep in the nap time.

A nap evaluation system of another aspect of the present disclosure includes the nap evaluation device and a display configured to present an evaluation result by the evaluation unit.

A nap evaluation method of still another aspect of the present disclosure includes a first acquisition process, a second acquisition process, and an evaluation process. The first acquisition process is a process of acquiring time information regarding a time during which a user is napping. The second acquisition process is a process of acquiring a sleep stage of the user who is napping. The evaluation process is a process of evaluating a quality of a nap in accordance with the time information and the sleep stage. In the evaluation process, an evaluation is made that the lower a ratio of a second sleep time to a first sleep time is, the higher the quality of the nap. The first sleep time is a sleep time in light sleep during which the sleep stage is relatively light in a nap time from a start to an end of the nap. The second sleep time is a sleep time in deep sleep during which the sleep stage is relatively deep in the nap time.

A program according to yet another aspect of the present disclosure is a program configured to cause one or more processors to execute the nap evaluation method.

### BRIEF DESCRIPTION OF DRAWINGS

FIG. 1 is a schematic system configuration diagram of a nap evaluation system including a nap evaluation device according to an embodiment of the present disclosure;

FIG. 2 is a graph of a time variation of a sleep state of a user for whom the nap evaluation device evaluates the quality of a nap; and

FIG. 3 is a flowchart illustrating operation of the nap evaluation device.

### DESCRIPTION OF EMBODIMENTS

### Embodiment

(1) Overview

A nap evaluation device according to the present embodiment and a nap evaluation system including the nap evaluation device will be described below with reference to the drawings.

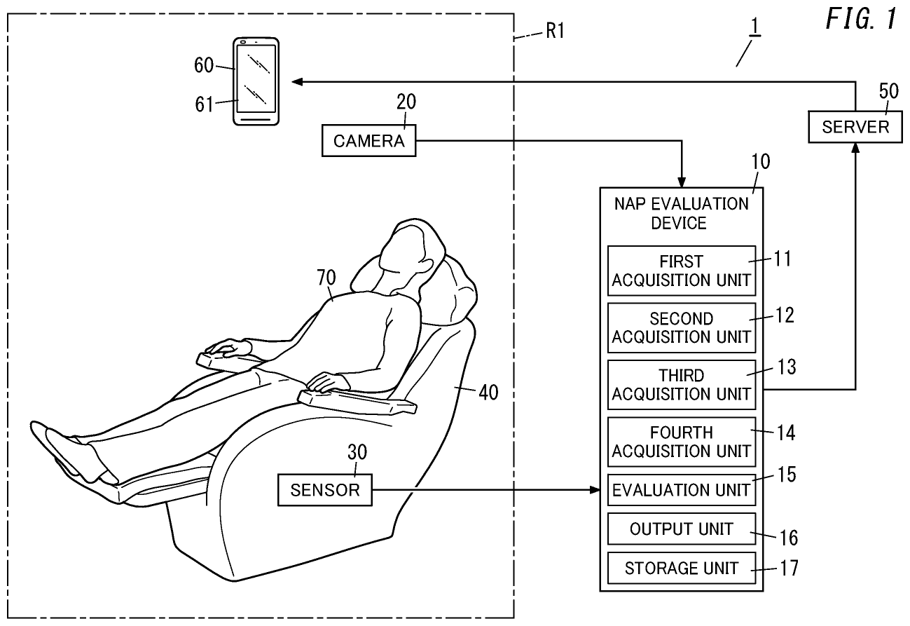

As illustrated in FIG. 1, a nap evaluation device 10 of the present embodiment includes a first acquisition unit 11, a second acquisition unit 12, and an evaluation unit 15.

The first acquisition unit 11 acquires time information regarding a time during which a user 70 is napping.

The second acquisition unit 12 acquires a sleep stage of the user 70 who is napping.

The evaluation unit 15 evaluates a quality of a nap in accordance with the time information and the sleep stage. The lower a ratio of a second sleep time to a first sleep time is, the higher the evaluation unit 15 evaluates a quality of the nap. The first sleep time is a sleep time in light sleep during which the sleep stage is relatively light in a nap time from a start to an end of the nap. The second sleep time is a sleep time in deep sleep during which the sleep stage is relatively deep in the nap time.

As used herein, the “user” is a person for whom the nap evaluation device 10 evaluates the quality of a nap. The “nap” refers to shorter sleep than usual (e.g., nocturnal) sleep and is taken for elimination of sleepiness, recovery from fatigue, or improvement of performance. The “nap time” is a time from a timing at which a user starts napping to a timing at which the user ends the napping. The nap time may include a time in which a user is in a wakefulness state before the user falls asleep and after the user awakens.

A sleep state is, for example, classified, based on the scoring system for sleep stages (Rechtschaffen & Kales, 1968), into REM sleep and non-REM sleep, and the non-REM sleep is further classified into four stages (sleep stages 1 to 4). Here, of the four sleep stages 1 to 4, light sleep during which the sleep stage is relatively light includes at least the sleep stage 2, and deep sleep during which the sleep stage is relatively deep includes at least the sleep stage 3. As described above, the sleep stage 2 is a sleep stage which is second lightest of four sleep stages 1 to 4 into which non-REM sleep is classified from light to deep sleep. Note that in the case of a short-time nap, deep sleep as the sleep stage 4 is less likely to occur, but the deep sleep may include the sleep stage 3 and the sleep stage 4. Note that the sleep stages may be based on classification by American Academy of Sleep Medicine (AASM). Alternatively, the sleep stages may be based on another classification correlated with the scoring system for sleep stages by Rechtschaffen & Kales or the classification by the AASM and may be estimated based on, for example, biological information such as body motion, heart rate, or respiration rate.

Here, the nap evaluation device 10 of the present embodiment evaluates an effective nap taken in a short nap time, for example, in a work break or the like as a high-quality nap. Moreover, if the nap time is extended, and deep sleep occurs during the nap, sleep inertia is more likely to occur after the end of the nap, which may increase a time until the user becomes awake after the nap. The lower the ratio of a second sleep time in the deep sleep to a first sleep time in the light sleep, the higher the evaluation unit 15 evaluates the quality of the nap, and therefore, the evaluation unit 15 can evaluate a nap which is effective for elimination of sleepiness and recovery from fatigue and which thereafter rapidly produces wakefulness as being high-quality. Thus, a nap evaluation device configured to evaluate the quality of the nap can be provided.

Moreover, the nap evaluation system 1 of the present embodiment includes the nap evaluation device 10 and a display (a portable information terminal 60). The nap evaluation device 10 outputs an evaluation result by the evaluation unit 15 to the display (the portable information terminal 60) via a server 50. The display (the portable information terminal 60) presents the evaluation result having been output from the nap evaluation device 10 and being the evaluation result by the evaluation unit 15.

The nap evaluation system 1 includes the nap evaluation device 10, and the display presents the evaluation result by the evaluation unit 15, and therefore, the nap evaluation system 1 including the nap evaluation device 10 configured to evaluate the quality of the nap can be provided.

(2) Details

The nap evaluation device 10 according to the present embodiment and the nap evaluation system 1 will be described in detail below with reference to FIGS. 1 to 3.

The nap evaluation device 10 of the present embodiment is used to evaluate, for example, the quality of a nap taken by the user 70 who naps in a recliner 40 for napping. Note that a place where the user 70 takes a nap is not limited to the recliner 40 but may be, for example, a bed for napping or may accordingly be changed. The environment (e.g., lighting, temperature, airflow, scent, and sound) in a nap room R1 in which the recliner 40 is placed is controlled by an environment control apparatus (e.g., a lighting fixture, an air conditioner, and an audio apparatus) so that the user 70 can comfortably take a nap. The nap evaluation device 10 is used to evaluate how the environment control of the nap room R1 by the environment control apparatus changes the quality of the nap, and the evaluation result by the nap evaluation device 10 may be fed back to the environment control apparatus.

In the nap room R1, a camera 20 for capturing motion of the face or the entire body of the user 70 who is napping in the recliner 40 is installed.

Moreover, to the recliner 40, a sensor 30 for collecting pieces of biological information and the like on the user 70 is attached. The pieces of biological information collected by the sensor 30 may include at least one of, for example, the pulse wave, heartbeat, heart's electrical potential, body motion, neural oscillation, ocular potential, mentalis potential, body temperature, and respiration rate of the user 70. The sensor 30 may include a radar biosensor for sensing, for example, subtle motion of the skin surface of the user 70 to sense at least one of the pulse wave, heartbeat, respiration rate and the like in a non-contact manner. The sensor 30 may include a biosensor for sensing at least one of the pulse wave, heart's electrical potential, respiration rate, body temperature, and the like by an electrode attached to, for example, the body surface of the user 70. The sensor 30 may include a temperature sensor for sensing infrared rays emitted from the body of the user 70 to sense the body temperature of the user 70. Moreover, the sensor 30 may include a lie-down detection sensor for sensing by a pressure sensitive sensor, for example, pressure applied to the recliner 40 to detect whether or not the user 70 is lying. Note that the sensor 30 may be disposed in the vicinity of the recliner 40 such that the pieces of biological information on the user 70 are collectable, or the sensor 30 may be a wearable sensor for collecting the pieces of biological information on the user 70 in a state where the sensor 30 is worn by the user 70.

(2.1) Nap Evaluation Device

The configuration of the nap evaluation device 10 will be described with reference to FIG. 1.

As described above, the nap evaluation device 10 includes the first acquisition unit 11, the second acquisition unit 12, and the evaluation unit 15. Moreover, the nap evaluation device 10 further includes a third acquisition unit 13, a fourth acquisition unit 14, an output unit 16, and a storage unit 17.

The nap evaluation device 10 includes, for example, a computer system. The computer system includes one or more processors and one or more memory elements as principal hardware components. The one or more processors execute a program(s) stored in the one or more memory elements of the computer system, thereby implementing functions as the nap evaluation device 10. The program(s) may be stored in advance in the one or more memory elements of the computer system. Alternatively, the program(s) may also be downloaded through an electric communication network or may be distributed after having been recorded in a non-transitory storage medium such as a memory card, an optical disc, or a hard disk drive, any of which is readable for the computer system.

The first acquisition unit 11 acquires time information regarding a time during which a user 70 is napping. The first acquisition unit 11 acquires a determination result from the lie-down detection sensor included in the sensor 30, that is, acquires a nap start time which is a timing (a time point) at which the user 70 lies (lies down) in the recliner 40 and a nap end time which is a timing at which the user 70 leaves the recliner 40. Here, the nap start time represents a time point at which a nap is started, specifically, a time point at which the user 70 lies down in the recliner 40, that is, the user is not sleeping at this time point. Moreover, the nap end time represents a time point at which the nap is ended, that is, the user is already awake at this time point. Note that a switch may be disposed in the vicinity of the recliner 40, and the user gives an operation to the switch at the time of starting or ending the nap such that the switch inputs to the first acquisition unit 11 a signal with reference to which the first acquisition unit 11 may acquire the nap start time and the nap end time. Alternatively, the first acquisition unit 11 may process an image captured by the camera 20 to detect whether or not the user 70 is lying in the recliner 40, thereby obtaining the nap start time or the nap end time. Still alternatively, the first acquisition unit 11 may process the image captured by the camera 20 to detect whether the eyes of the user 70 lying in the recliner 40 are open or closed and may acquire a timing at which the user 70 closes his/her eyes as the nap start time and a timing at which the user 70 opens his/her eyes as the nap end time.

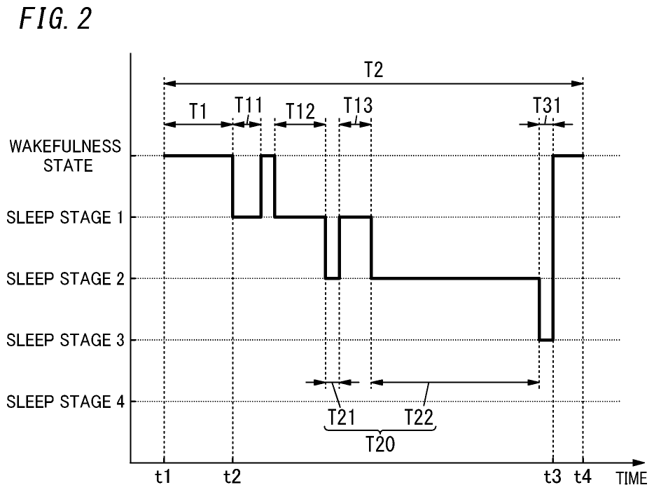

The second acquisition unit 12 is configured to acquire a sleep stage of the user who is napping. Specifically, the second acquisition unit 12 processes the image captured by the camera 20 to detect the body motion of the user 70 who is napping. Moreover, the second acquisition unit 12 acquires, from the sensor 30, biological information (information regarding at least one of, for example, the pulse wave, heartbeat, heart's electrical potential, body motion, neural oscillation, ocular potential, mentalis potential, body temperature, and respiration rate) on the user 70 who is napping. The second acquisition unit 12 determines, based on the biological information acquired at prescribed time intervals from the sensor 30 in a time period from the nap start time to the nap end time, the sleep stage of the user 70 in accordance with, for example, the scoring system for sleep stages, and thereby, the second acquisition unit 12 acquires the sleep stage of the user 70. That is, the sleep stage is a value determined with reference to the biological information on the user 70. The second acquisition unit 12 stores time information at the time of the determination of the sleep stage and information on a determination result of the sleep stage in association with each other in the storage unit 17. In this embodiment, FIG. 2 shows an example of a result of determination (acquisition) of the sleep stage of the user 70 by the second acquisition unit 12 in a time period from a nap start time t1 to a nap end time t4. In the example shown in FIG. 2, it is determined that the user 70 is in the wakefulness state for a while after the nap start time t1 and that the user 70 enters a sleep state at the time point t2. The sleep state thereafter changes over time, and it is determined that the user 70 rapidly awakens from the sleep state at the time point t3 by a wake-up signal, such as a sound of a bell, and the nap ends at the nap end time t4. Note that only during the time period from the nap start time t1 to the nap end time t4, the second acquisition unit 12 may determine the sleep state of the user 70 who is sleeping. Moreover, the second acquisition unit 12 may determine, based on the biological information acquired at irregular timings from the sensor 30 in the time period from the nap start time t1 to the nap end time t4, the sleep stage of the user 70.

The third acquisition unit 13 acquires a sleep onset time until the user 70 falls asleep. Specifically, the third acquisition unit 13 acquires, as a sleep onset time T1, an elapsed time from the nap start time t1 acquired by the first acquisition unit 11 to the time point t2 at which the second acquisition unit 12 determines that the user 70 is in the sleep state (e.g., a sleep state corresponding to the sleep stage 1). Note that the third acquisition unit 13 is not an essential configuration of the nap evaluation device 10 and may accordingly be omitted.

The fourth acquisition unit 14 acquires wakefulness information regarding the level of wakefulness of the user 70. The fourth acquisition unit 14 processes the image captured by the camera 20, for example, in a time period from when the user 70 sleeping in the recliner 40 awakens and to when the user 70 leaves the recliner 40, thereby acquiring the wakefulness information regarding the level of wakefulness of the user 70 from facial expressions or the like of the user 70. The fourth acquisition unit 14 processes the image captured by the camera 20 to sense, as the facial expressions, the degree of opening of the eyelids, and the cycle and the number of blinks of the user 70, and the fourth acquisition unit 14 determines, based on these facial expressions, the degree of the wakefulness of the user 70. For example, the larger the number of times of closing the eyelids is, the lower the degree of opening of the eyelids is, or the larger the number of blinks is, the lower the fourth acquisition unit 14 determines the level of wakefulness of the user 70. In contrast, the smaller the number of times of closing the eyelids is, the higher the degree of opening of the eyelids is, or the smaller the number of blinks is, the higher the fourth acquisition unit 14 determines the level of wakefulness of the user 70. Note that the fourth acquisition unit 14 is not an essential configuration of the nap evaluation device 10 and may accordingly be omitted.

The evaluation unit 15 evaluates, based on the time information acquired by the first acquisition unit 11 and the sleep stage acquired by the second acquisition unit 12, the quality of the nap that the user 70 has taken.

The evaluation unit 15 obtains, based on the time information, the nap time from when the nap starts to when the nap ends. In the example shown in FIG. 2, the evaluation unit 15 obtains, as a nap time T2 (minute), a time from when the nap starts to when the nap ends, that is, the time period from the nap start time t1 to the nap end time t4.

Moreover, the evaluation unit 15 obtains, based on the time information and the sleep stage, a sleep time in light sleep during which the sleep stage is relatively light as the first sleep time in the nap time T2. In the present embodiment, the evaluation unit 15 obtains, as a first sleep time T20 (minute), a sleep time (the total time of times T21 and T22) in the sleep stage 2. Note that in the sleep stage 1, sleep is lighter and the effect of elimination of sleepiness or recovery from fatigue is thus less than in the sleep stage 2, but the evaluation unit 15 may include the sleep times T11, T12, and T13 in the sleep stage 1 into the first sleep time in light sleep.

Moreover, the evaluation unit 15 obtains, as a second sleep time T31 (minute), a sleep time in deep sleep during which the sleep stage is relatively deep (e.g., the sleep stage 3 and the sleep stage 4) in the nap time T2. In the case of a short-time nap, the sleep state of the sleep stage 4 is less likely to occur, and therefore, the evaluation unit 15 obtains, in practice, the sleep time in the sleep stage 3 as the second sleep time.

The evaluation unit 15 obtains the nap time T2, the first sleep time T20, and the second sleep time T31, and then, the shorter the nap time T2 is and the smaller the ratio of the second sleep time T31 to the first sleep time T20 is, the higher the evaluation unit 15 evaluates the quality of the nap. For example, the evaluation unit 15 compares the nap time T2, the first sleep time T20, and the second sleep time T31 to respective reference values to obtain an evaluation value E1 representing the quality of the nap. If the nap time is too long, the time until the user returns to the wakefulness state after the nap tends to increase, and therefore, the reference value of the nap time T2 is set to, for example, 20 minutes. Moreover, based on a study (“A short nap as countermeasure against afternoon sleepiness”, Physiological Psychology and Psychophysiology, Volume 25, No. 1, pp. 45-59, 2007) that the sleep stage 2 of three minutes is effective to suppress sleepiness, the reference value of the first sleep time T20 is set to, for example, 3 minutes. Further, the sleep time in deep sleep is desirably zero or as small value as possible, and therefore, the reference value of the second sleep time T31 is set to, for example, 0 minutes. In this embodiment, the evaluation unit 15 obtains, based on the following formula 1, the differences of the nap time T2, the first sleep time T20, and the second sleep time T31 from their respective reference values and calculates the total value of these differences, thereby obtaining the evaluation value E1 representing the quality of the nap. The evaluation unit 15 stores the evaluation value E1 thus calculated in the storage unit 17.

E1=(20−T2)+(T20−3)+(0−T31) [Formula 1]

In this case, the shorter the nap time T2 is than 30 minutes, the longer the first sleep time T20 is than 3 minutes, and the shorter the second sleep time T31 can be, the larger the evaluation value E1, and thus, the nap can be evaluated as being good-quality. Note that the reference values of the nap time T2, the first sleep time T20, and the second sleep time T31 are not limited to fixed values but may be, for example, previous values or average values.

Moreover, the evaluation unit 15 may multiply comparison results obtained by comparing the nap time T2, the first sleep time T20, and the second sleep time T31 to their respective reference values by coefficients al, bl, and cl respectively to obtain an evaluation value E2 weighted. In this case, the evaluation value E2 is obtained from the following formula 2.

E2=A1×(20−T2)+B1×(T20−3)+C1×(0−T31) [Formula 2]

In this case, the coefficient of an item, which significantly affects the quality of the nap, of three items, namely, the nap time T2, the first sleep time T20, and the second sleep time T31 is set to a value larger than each of coefficients of the other items, and thereby, the evaluation value E2 which more accurately represents the quality of the nap is obtainable. Note that the coefficients al, bl, and cl are at least set to values respectively depending on the contribution ratios of the nap time T2, the first sleep time T20, and the second sleep time T31 and may thus be accordingly changed. Note that the evaluation unit 15 may evaluate the quality of sleep in accordance with only the ratio of the second sleep time T31 to the first sleep time T20, and the ratio of the second sleep time T31 to the first sleep time T20 is small, and therefore, it is possible to determine that an effective nap has been taken.

Moreover, in addition to the determination described above, the evaluation unit 15 may further make an evaluation that the shorter the sleep onset time T1 acquired by the third acquisition unit 13 is, the higher the quality of the nap. Even when the nap time T2 is the same, the first sleep time T20 which is the sleep time in the second sleep stage 2 tends to increase as the sleep onset time T1 decreases. Here, when the reference value of the sleep onset time T1 is set to TA1 (e.g., ca. 5 minutes), the evaluation unit 15 may obtain, as an evaluation value of the quality of the nap, a value obtained by adding a value (TA1−T1) to the evaluation value E1 or E2. The evaluation value increases as the sleep onset time T1 shortens, and therefore, the user 70 can identify, with reference to this evaluation value, whether the quality of the nap is good or bad.

Moreover, in addition to the determination described above, the evaluation unit 15 may further make an evaluation that the longer a continuation time for which the sleep stage 2 continues is, the higher the quality of the nap. The occurrence of deep sleep in the nap is undesirable, and therefore, a time of the sleep stage 2 corresponding to the deepest sleep in the light sleep is preferably long. Moreover, in the case of an instable sleep state in which the sleep state of the user 70 frequently changes between the sleep stage 1 and the sleep stage 2, it is determined that no good-quality sleep is obtained, and therefore, the continuation time for which the sleep stage 2 continues (in the example shown in FIG. 2, time T22) is preferably long. Thus, the evaluation unit 15 may obtain, as the evaluation value of the quality of the nap, a value obtained by adding, to the evaluation value E1 or E2, a difference between a maximum value (time T22) of the continuation time of the sleep stage 2 and the reference value (e.g., 3 minutes). Alternatively, the evaluation unit 15 may obtain, as the evaluation value of the quality of the nap, a value obtained by adding, to the evaluation value E1 or E2, a value obtained by multiplying a difference between a maximum value (time T22) of the continuation time of the sleep stage 2 and the reference value (e.g., 3 minutes) by a coefficient. The longer the continuation time of the sleep stage 2 is, the larger the evaluation values, and therefore, the user 70 can identify, with reference to this evaluation value, whether the quality of the nap is good or bad.

Moreover, in addition to the determination described above, the evaluation unit 15 may further make an evaluation, based on the wakefulness information acquired by the fourth acquisition unit 14, that the shorter the recovery time in which the user 70 becomes awake after the end of the nap is, the higher the quality of the nap. In this case, if the user 70 takes a nap effective for elimination of sleepiness or recovery from fatigue, the recovery time, in which the user 70 becomes awake after the end of the nap, is supposed to decrease. The evaluation unit 15 estimates, based on the wakefulness information (e.g., facial expressions of the user 70) acquired by the fourth acquisition unit 14, the recovery time, in which the user 70 becomes awake after the end of the nap. Then, the evaluation unit 15 may obtain, as the evaluation value of the quality of the nap, a value obtained by adding, to the evaluation value E1 or E2, a value obtained by subtracting the recovery time from the reference value (e.g., several minutes) of the recovery time. The evaluation value increases as the recovery time shortens, and therefore, the user 70 can identify, with reference to this evaluation value, whether the quality of the nap is good or bad.

Moreover, in addition to the determination described above, the evaluation unit 15 may make an evaluation that the shorter the time for which the user 70 is in the wakefulness state between the start to the end of the nap is, the higher the quality of the nap. As the time for which the user 70 is in the wakefulness state during the nap increases, the time for which the user 70 is in the sleep state decreases, and therefore, the quality of the nap decreases. In other words, if the time for which the user 70 is in the sleep state is the same, the nap time increases as the time for which the user 70 is in the wakefulness state during the nap, and therefore, the quality of the nap decreases. Thus, the evaluation unit 15 makes the evaluation that the shorter the time for which the user 70 is in the wakefulness state between the start to the end of the nap is, the higher the quality of the nap, and thereby, the evaluation unit 15 can evaluate the quality of the nap with further improved accuracy.

The storage unit 17 includes, for example, electrically rewritable nonvolatile memory such as Electrically Erasable Programmable Read Only Memory (EEPROM). The storage unit 17 stores, for example, time information acquired by the first acquisition unit 11 during the nap and time information and information on evaluation result when the second acquisition unit 12 evaluates the sleep stage. Moreover, the storage unit 17 stores the evaluation result, which is obtained by evaluating the quality of the nap, by the evaluation unit 15. In this case, the nap evaluation device 10 may acquire the identification information of the user 70 who is napping, for example, from the portable information terminal 60 carried by the user 70, and the nap evaluation device 10 may store the evaluation result of the quality of the nap and the identification information of the user 70 associated with each other in the storage unit 17.

The output unit 16 outputs the evaluation result by the evaluation unit 15. The output unit 16 outputs the evaluation result by the evaluation unit 15 via the server 50 to the portable information terminal 60, such as a smartphone, carried by the user 70. The evaluation result output from the output unit 16 is not limited to the evaluation value E1 or E2 but may include at least one piece of information of pieces of information regarding the sleep onset time T1, the nap time T2, the continuation time of the sleep stage 2, and wakefulness information on the level of wakefulness of the user 70. The evaluation result by the evaluation unit 15 is output from the output unit 16 and is then presented to the user 70 by the portable information terminal 60. Thus, the user 70 can identify the quality of the nap with reference to the information presented by the portable information terminal 60.

Here, the output unit 16 may output the evaluation result by the evaluation unit 15 and a past evaluation result of the user 70 acquired from the storage unit 17 to the portable information terminal 60. This enables the portable information terminal 60, which is a display, to present (e.g., display on a display monitor 61) the evaluation result by the evaluation unit 15 and the past evaluation result of the user 70 acquired from the storage unit 17 in a comparative manner. Moreover, the output unit 16 may display a result of comparison between a present evaluation result and a previous evaluation result (e.g., a result of comparison in terms of the length of the continuation time of the sleep stage 2) on the display monitor 61 of the portable information terminal 60. Moreover, when the storage unit 17 stores evaluation results of naps taken by a plurality of users for the respective users, the output unit 16 may display (present) the evaluation result by the evaluation unit 15 and the evaluation result of another user stored in the storage unit 17 in a comparative manner on the display monitor 61 of the portable information terminal 60.

Moreover, the output unit 16 may output the evaluation result by the evaluation unit 15 and the past evaluation result of another user acquired from the storage unit 17 to the portable information terminal 60. This enables the portable information terminal 60, which is a display, to present the evaluation result by the evaluation unit 15 and the past evaluation result of another user acquired from the storage unit 17 in a comparative manner.

(2.2) Nap Evaluation System

Next, the nap evaluation system 1 will be described with reference to FIG. 1.

The nap evaluation system 1 includes the nap evaluation device 10 and the portable information terminal 60 (display) for presenting the evaluation result by the evaluation unit 15. The portable information terminal 60 is a smartphone portable by the user 70 who is to take a nap in the recliner 40. Note that the portable information terminal 60 is not limited to the smartphone but may be a tablet computer terminal or a wearable terminal which is to be worn by the user 70.

The nap evaluation device 10 includes a communication unit configured to communicate with the server 50, for example, via a communication network such as the Internet. The output unit 16 of the nap evaluation device 10 outputs evaluation results obtained by evaluation of the nap of the user 70 by the evaluation unit 15 (e.g., the evaluation value E1 or E2, the sleep onset time T1, the nap time T2, and the continuation time of the sleep stage 2) from the communication unit to the server 50. The network address (e.g., electronic mail address or IP address) of the portable information terminal 60 carried by the user 70 is registered with the server 50. When the server 50 receives an evaluation result of a nap of the user 70 from the nap evaluation device 10, the server 50 outputs the evaluation result, for example, via a mobile communication network, to the portable information terminal 60 carried by the user 70.

When the portable information terminal 60 receives the evaluation result of the nap transmitted from the nap evaluation device 10, the portable information terminal 60 displays the evaluation result of the nap on the display monitor 61. For example, the portable information terminal 60 displays the evaluation value (e.g., evaluation value E1 or E2) obtained by the evaluation unit 15 on the display monitor 61. The user 70 who took the nap views the evaluation result displayed on the display monitor 61 of the portable information terminal 60 to visually recognize the quality of the nap. Note that the portable information terminal 60 may display, on the display monitor 61, an evaluation comment (e.g., “nap time is too long”, “sleep was light”, or “sleep was deep”) created based on the evaluation result of the nap transmitted from the nap evaluation device 10. Moreover, the output unit 16 may output the evaluation result by the evaluation unit 15 in voice, for example, from a loudspeaker of the portable information terminal 60, or the output unit 16 may display the evaluation result on the display monitor 61 and output the evaluation result as an audio output from the loudspeaker.

Note that the portable information terminal 60 may display (present), based on a present evaluation result by the evaluation unit 15 transmitted from the nap evaluation device 10 and a past evaluation result, an evaluation result of the most recent nap taken by the user 70 and an evaluation result of a nap taken before the most recent nap in a comparative manner. Here, “displaying in a comparative manner” may be a display mode in which the evaluation result of the most recent nap and the evaluation result of the nap taken before the most recent nap are displayed in one screen or a display mode in which the most recent evaluation value and the difference between the most recent evaluation value and the evaluation value before the most recent evaluation value are displayed together. Thus, the user 70 can identify the evaluation value of the most recent nap in a manner comparative to the evaluation value of the nap taken before the most recent nap.

Moreover, when the evaluation result of the user 70 who has taken a nap this time and a past evaluation result of another user are transmitted from the nap evaluation device 10 to the portable information terminal 60, the portable information terminal 60 may display (present) the evaluation result of the user 70 and the evaluation result of the another user in a comparative manner. Thus, the user 70 can identify the evaluation value of the nap taken by himself/herself in a manner comparative to an evaluation value of a nap taken by another user.

(2.3) Operation

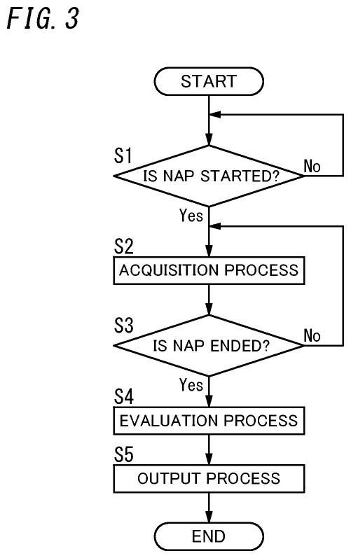

Operation of the nap evaluation device 10 of the present embodiment will be described with reference to FIG. 3.

The first acquisition unit 11 of the nap evaluation device 10 regularly acquires a detection result from the lie-down detection sensor, and based on the detection result from the lie-down detection sensor, the first acquisition unit 11 determines whether or not the nap (actually, preparation for a nap) is started (S1).

When acquiring, from the lie-down detection sensor, a detection result that the user 70 lies down, the first acquisition unit 11 determines that the user 70 starts napping (actually preparation for napping) (S1: Yes).

When the nap evaluation device 10 determines that the user 70 starts napping, each of the first acquisition unit 11, the second acquisition unit 12, the third acquisition unit 13, and the fourth acquisition unit 14 of the nap evaluation device 10 performs an acquisition process (S2). Here, the second acquisition unit 12 determines the sleep stage of the user 70 in accordance with the biological information which is on the user 70 and which is input from the sensor 30, and pieces of information acquired by the first acquisition unit 11, the second acquisition unit 12, the third acquisition unit 13, and the fourth acquisition unit 14 are stored in the storage unit 17.

Moreover, the first acquisition unit 11 determines whether or not the user 70 ends the nap (actually, leaves the recliner) in accordance with the detection result acquired from the lie-down detection sensor (S3), and if the nap is not ended (S3: No), the first acquisition unit 11 repeats the acquisition process in S2.

When the first acquisition unit 11 thereafter acquires, from the lie-down detection sensor, a detection result that the user 70 has left the recliner, the first acquisition unit 11 determines that the user 70 has ended the nap (S3: Yes).

When the nap ends, the evaluation unit 15 evaluates the quality of the nap in accordance with the pieces of information acquired by the first acquisition unit 11, the second acquisition unit 12, the third acquisition unit 13, and the fourth acquisition unit 14 during the nap (S4). Then, the output unit 16 transmits the evaluation result by the evaluation unit 15 from the communication unit via the server 50 to the portable information terminal 60. When the portable information terminal 60 receives the evaluation result of the nap from the nap evaluation device 10, the portable information terminal 60 displays the evaluation result of the nap on the display monitor 61, and the user 70 can identify the quality of the nap of the user 70 with reference to the contents displayed on the display monitor 61.

(3) Variation

The embodiment described above is merely an example of various embodiments of the present disclosure. Rather, the embodiment described above may be readily modified in various manners depending on a design choice or any other factor without departing from the scope of the present disclosure. Moreover, a function similar to the nap evaluation device 10 may be implemented by a nap evaluation method, a computer program, a non-transitory storage medium in which a program is recorded, or the like. A nap evaluation method according to an aspect includes a first acquisition process, a second acquisition process, and an evaluation process. The first acquisition process includes acquiring time information regarding a time during which a user 70 is napping. The second acquisition process includes acquiring a sleep stage of the user 70 who is napping. The evaluation process includes evaluating a quality of a nap in accordance with the time information and the sleep stage. The lower a ratio of a second sleep time to a first sleep time is, the higher the quality of the nap is evaluated in the evaluation process. The first sleep time is a sleep time in light sleep during which the sleep stage is relatively light in a nap time from a start to an end of the nap. The second sleep time is a sleep time in deep sleep during which the sleep stage is relatively deep in the nap time. A (computer) program according to one aspect is a program configured to cause one or more processors to execute the nap evaluation method (the first acquisition process, the second acquisition process, and the evaluation process).

Variations of the embodiment described above will be described below. The variations described below are applicable accordingly in combination.

The nap evaluation device 10 in the present disclosure includes the computer system. The computer system includes a processor and a memory element as principal hardware components. The functions as the nap evaluation device 10 according to the present disclosure may be implemented by making the processor execute a program stored in the memory element of the computer system. The program may be stored in the memory element of the computer system in advance, may be provided via a telecommunications network, or may be provided as a non-transitory recording medium such as a computer system-readable memory card, optical disc, or hard disk drive storing the program. The processor of the computer system may be made up of a single or a plurality of electronic circuits including a semiconductor integrated circuit (IC) or a largescale integrated circuit (LSI). The integrated circuit such as IC or LSI mentioned herein may be referred to in another way, depending on the degree of the integration and includes integrated circuits called system LSI, very-large-scale integration (VLSI), or ultra-large-scale integration (ULSI). Optionally, a field-programmable gate array (FPGA) to be programmed after an LSI has been fabricated or a reconfigurable logic device allowing the connections or circuit sections inside of an LSI to be reconfigured may also be adopted as the processor. The plurality of electronic circuits may be collected on one chip or may be distributed on a plurality of chips. The plurality of chips may be collected in one device or may be distributed in a plurality of devices. As mentioned herein, the computer system includes a microcontroller including one or more processors and one or more memory elements. Thus, the microcontroller is also composed of one or more electronic circuits including a semiconductor integrated circuit or a large-scale integrated circuit.

Collecting the plurality of functions of the nap evaluation device 10 in one housing is not an essential configuration of the nap evaluation device 10. The components of the nap evaluation device 10 may be distributed in a plurality of housings. Alternatively, at least some functions (e.g., the evaluation unit 15) of the nap evaluation device 10 may be implemented as a cloud computing system as well.

In contrast, at least some of functions of the nap evaluation system 1 distributed in a plurality of devices in the embodiment described above may be collected in one housing. In the embodiment described above, at least some functions of the nap evaluation system 1 which are distributed in the nap evaluation device 10 and the display (the portable information terminal 60) may be collected in one housing.

In the embodiment described above, the nap evaluation device 10 may receive biological information from a wearable sensor or the like carried by the user 70 via the mobile communication network at a timing at which a prescribed time elapses since the end of the nap (the nap end time t4). Here, the prescribed time is determined depending on a timing at which, for example, whether or not the effect of the nap persists is desirably evaluated. The prescribed time is set to, for example, about 1 to 2 hours, but the time may accordingly be changed. Moreover, the biological information received from the wearable sensor is wakefulness information regarding the level of wakefulness of the user 70 and may include at least one piece of information of, for example, the pulse wave, heartbeat, heart's electrical potential, body motion, neural oscillation, ocular potential, mentalis potential, body temperature, and respiration rate of the user 70. In this case, the evaluation unit 15 may obtain the wakefulness of the user 70 with reference to the biological information received from the wearable sensor or the like. If the wakefulness of the user 70 at this timing is higher than a prescribed threshold, that is, if the user 70 feels less sleepy, the evaluation unit 15 determines that the nap taken by the user 70 is good-quality. Thus, the evaluation unit 15 may evaluate the quality of the nap further with reference to the wakefulness information at a timing at which the prescribed time elapses since the end of the nap, and the evaluation unit 15 can evaluate the quality of the nap in accordance with the degree of persistence of the effect of elimination of sleepiness or recovery from fatigue by taking the nap.

Moreover, the nap evaluation device 10 may receive biological information from the wearable sensor or the like carried by the user 70 via the mobile communication network at a timing at which the user 70 gets usual sleep after the nap ends. Here, the biological information received from the wearable sensor or the like is information regarding the quality of the usual sleep and may include at least one piece of information of, for example, the pulse wave, heartbeat, heart's electrical potential, body motion, neural oscillation, ocular potential, mentalis potential, body temperature, and respiration rate. In this case, the evaluation unit 15 evaluates the quality of the usual sleep in accordance with the biological information received from the wearable sensor or the like. The evaluation unit 15 obtains, as indexes for evaluation of the quality of the usual sleep, indexes of, for example, a sleep onset time (sleep onset latent time) in the usual sleep, the ratio (deep sleep ratio) of the time in the deep sleep to the total sleep time, and the number of times of wake-up after sleep onset. Here, it is assumed that a high quality of the previous nap results in a good quality of the usual sleep taken thereafter, and therefore, the evaluation unit 15 may, based on the indexes for evaluation of the quality of the usual sleep, make an evaluation that the higher the quality of the usual sleep is, the higher the quality of the previous nap. Thus, the nap evaluation device 10 can evaluate the quality of the previous nap in consideration of the quality of the usual sleep after the nap.

In the embodiment described above, the shorter the nap time is, and the higher the ratio of the first sleep time in light sleep to the nap time is, the higher the evaluation unit 15 may evaluate the quality of the nap, and thus, the evaluation unit 15 can evaluate that a short-time nap with a high ratio of the light sleep is high-quality. Moreover, the shorter the nap time is, and the lower the ratio of the second sleep time in deep sleep to the nap time is, the higher the evaluation unit 15 may evaluate the quality of the nap, and thus, the evaluation unit 15 can evaluate that a short-time nap with a low ratio of the deep sleep is high-quality.

In the embodiment, the display is the portable information terminal 60 carried by the user 70, but the display may be a display device or the like provided to the recliner 40 or the nap evaluation device 10.

In the embodiment, the portable information terminal 60, which is the display, presents the evaluation result by the evaluation unit 15 to the user 70 who has taken a nap, but a user to whom the display presents the evaluation result is not limited to the user 70 who takes a nap. For example, the display may present the evaluation result by the evaluation unit 15 to a user as is an evaluator who evaluates the quality of the nap taken by the user 70 by using the nap evaluation device 10.

Moreover, in the embodiment described above, the nap time does not necessarily include a time (time from the nap start time t1 to the time point t2) in which a user is in the wakefulness state before the user falls asleep or a time (time from the time point t3 to the nap end time t4) in which the user is in the wakefulness state after the user awakens. That is, the nap time may be a time from when a user falls asleep to when the user awakens (time from the time point t2 to the time point t3).

### SUMMARY

As described above, a nap evaluation device (10) of a first aspect includes a first acquisition unit (11), a second acquisition unit (12), and an evaluation unit (15). The first acquisition unit (11) is configured to acquire time information regarding a time during which a user (70) is napping. The second acquisition unit (12) is configured to acquire a sleep stage of the user (70) who is napping. The evaluation unit (15) is configured to evaluate a quality of a nap in accordance with the time information and the sleep stage. The evaluation unit (15) makes an evaluation that the lower a ratio of a second sleep time (T31) to a first sleep time (T20) is, the higher the quality of the nap. The first sleep time (T20) is a sleep time in light sleep during which the sleep stage is relatively light in a nap time from a start to an end of the nap. The second sleep time (T31) is a sleep time in deep sleep during which the sleep stage is relatively deep in the nap time.

With this aspect, the lower the ratio of the second sleep time (T31) in the deep sleep to the first sleep time (T20) in the light sleep is, the higher the evaluation unit (15) evaluates the quality of the nap. Thus, this aspect provides the nap evaluation device (10) configured to evaluate the quality of the nap.

In a nap evaluation device (10) of a second aspect referring to the first aspect, the evaluation unit (15) makes an evaluation that the shorter the nap time is, the higher the quality of the nap.

Because a time for returning to a wakefulness state decreases as the nap time decreases, this aspect enables an evaluation to be made that the shorter the nap time is, the higher the quality of the nap.

In a nap evaluation device (10) of a third aspect referring to the first or second aspect, the evaluation unit (15) makes an evaluation that the shorter a time of a wakefulness state between a start to an end of the nap is, the higher the quality of the nap.

This aspect enables an evaluation to be made that the shorter the time of the wakefulness state during the nap is, the higher the quality of the nap.

In a nap evaluation device (10) of a fourth aspect referring to any one of the first to third aspects, the sleep stage is a value determined with reference to biological information on the user (70).

With this aspect, the second acquisition unit (12) acquires the sleep stage of the user (70) by determining the sleep stage with reference to the biological information on the user (70).

A nap evaluation device (10) of a fifth aspect referring to any one of the first to fourth aspects further includes a third acquisition unit (13) configured to acquire a sleep onset time (T1) which is a time until the user (70) falls asleep. The evaluation unit (15) further makes an evaluation that the shorter the sleep onset time (T1) is, the higher the quality of the nap.

It is assumed that as the sleep onset time (T1) decreases, the first sleep time in light sleep increases, and therefore, this aspect enables the quality of the nap to be evaluated based on the sleep onset time (T1).

In a nap evaluation device (10) of a sixth aspect referring to any one of the first to fifth aspects, the light sleep includes a sleep stage 2 which is second lightest of four sleep stages 1 to 4 into which non-REM sleep is classified from light to deep sleep. The evaluation unit (15) further makes an evaluation that the longer a continuation time for which the sleep stage 2 continues is, the higher the quality of the nap.

Here, if the sleep state of the user (70) corresponds to instable sleep in which the sleep state frequently varies between the sleep stage 1 and the sleep stage 2, the nap is assumed to be bad-quality. The longer a continuation time for which the sleep stage 2 continues is, the higher the evaluation unit (15) evaluates the quality of the nap, and thus, the evaluation unit (15) is configured to evaluate the quality of the nap further with reference to the continuation time of the sleep stage 2.

A nap evaluation device (10) of a seventh aspect referring to any one of the first to sixth aspects further includes a fourth acquisition unit (14) configured to acquire wakefulness information regarding a level of wakefulness of the user (70). The evaluation unit (15) makes, further based on the wakefulness information, an evaluation that the shorter the recovery time in which the user (70) becomes awake after the end of the nap is, the higher the quality of the nap.

In this case, if the user (70) takes a nap effective for elimination of sleepiness or recovery from fatigue, the recovery time in which the user (70) becomes awake after the end of the nap is assumed to decrease. This aspect enables the evaluation unit (15) to make an evaluation that the shorter the recovery time in which the user (70) becomes awake after the end of the nap is, the higher the quality of the nap.

In a nap evaluation device (10) of an eighth aspect referring to the seventh aspect, the evaluation unit (15) evaluates the quality of the nap further with reference to the wakefulness information at a timing at which the prescribed time elapses since the end of the nap.

In this case, it is assumed that if the user (70) takes a nap effective for elimination of sleepiness or recovery from fatigue, the user (70) feels less sleepy after the end of the nap. With this aspect, the evaluation unit (15) evaluates the quality of the nap with reference to the wakefulness information at the timing at which the prescribed time elapses since the end of the nap, and thus, this aspect provides a nap evaluation device (10) configured to more finely evaluate the quality of the nap.

A nap evaluation device (10) of a ninth aspect referring to any one of the first to eighth aspects further includes an output unit (16) configured to output an evaluation result by the evaluation unit (15).

This aspect enables the quality of the nap to be identified based on the evaluation result by the evaluation unit (15) output from the output unit (16).

The nap evaluation system (1) of the tenth aspect includes the nap evaluation device (10) of any one of the first to ninth aspects, and a display (60) configured to present an evaluation result by the evaluation unit (15).

With this aspect, the nap evaluation system (1) including the nap evaluation device (10) configured to evaluate the quality of the nap can be provided.

In a nap evaluation system (1) of an eleventh aspect referring to the tenth aspect, the display (60) is configured to present the evaluation result by the evaluation unit (15) and a past evaluation result acquired from a storage unit (17) configured to store evaluation results by the evaluation unit (15) in a comparative manner.

With this aspect, based on the information presented by the display (60), the evaluation result by the evaluation unit (15) is comparable with the past evaluation result.

A nap evaluation method of a twelfth aspect includes a first acquisition process, a second acquisition process, and an evaluation process. The first acquisition process is a process of acquiring time information regarding a time during which a user (70) is napping. The second acquisition process is a process of acquiring a sleep stage of the user (70) who is napping. The evaluation process is a process of evaluating a quality of a nap in accordance with the time information and the sleep stage. In the evaluation process, an evaluation is made that the lower a ratio of a second sleep time (T31) to a first sleep time (T20) is, the higher the quality of the nap. The first sleep time (T20) is a sleep time in light sleep during which the sleep stage is relatively light in a nap time from a start to an end of the nap. The second sleep time (T31) is a sleep time in deep sleep during which the sleep stage is relatively deep in the nap time.

This aspect enables the quality of the nap to be evaluated.

A program of a thirteenth aspect is configured to cause one or more processors to execute the nap evaluation method of the twelfth aspect.

This aspect enables the quality of the nap to be evaluated.

The aspects should not be construed as limiting, but various configurations (including variations) of the nap evaluation device (10) of the embodiment may be embodied by, for example, a nap evaluation method, a (computer) program, or a non-transitory recording medium in which a program is stored.

Note that constituent elements according to the second to ninth aspects are not essential constituent elements for the nap evaluation device (10) but may be omitted as appropriate. The configuration according to the eleventh aspect is not configurations essential for the nap evaluation system (1) and may thus be accordingly omitted.

### REFERENCE SIGNS LIST

1 Nap Evaluation System
10 Nap Evaluation Device
11 First Acquisition Unit
12 Second Acquisition Unit
13 Third Acquisition Unit
14 Fourth Acquisition Unit
15 Evaluation Unit
16 Output Unit
17 Storage Unit
60 Display
70 User
T1 Sleep Onset Time
T2 Nap Time
T20 First Sleep Time
T31 Second Sleep Time

1. A nap evaluation device, comprising:
a first acquisition unit configured to acquire time information regarding a time during which a user is napping;
a second acquisition unit configured to acquire a sleep stage of the user who is napping; and
an evaluation unit configured to evaluate a quality of a nap in accordance with the time information and the sleep stage,
the evaluation unit making an evaluation that the lower a ratio of a second sleep time to a first sleep time is, the higher the quality of the nap,
the first sleep time being a sleep time in light sleep during which the sleep stage is relatively light in a nap time from a start to an end of the nap, the second sleep time being a sleep time in deep sleep during which the sleep stage is relatively deep in the nap time.

2. The nap evaluation device of claim 1, wherein
the evaluation unit makes an evaluation that the shorter the nap time is, the higher the quality of the nap.

3. The nap evaluation device of claim 1,
the evaluation unit makes an evaluation that the shorter a time of a wakefulness state between a start to an end of the nap is, the higher the quality of the nap.

4. The nap evaluation device of claim 1, wherein
the sleep stage is a value determined with reference to biological information on the user.

5. The nap evaluation device of claim 1, further comprising a third acquisition unit configured to acquire a sleep onset time which is a time until the user falls asleep, wherein
the evaluation unit further makes an evaluation that the shorter the sleep onset time is, the higher the quality of the nap.

6. The nap evaluation device of claim 1, wherein
the light sleep includes a sleep stage 2 which is second lightest of four sleep stages 1 to 4 into which non-REM sleep is classified from light to deep sleep, and
the evaluation unit further makes an evaluation that the longer a continuation time for which the sleep stage 2 continues is, the higher the quality of the nap.

7. The nap evaluation device of claim 1, further comprising a fourth acquisition unit configured to acquire wakefulness information regarding wakefulness of the user, wherein
the evaluation unit makes, further based on the wakefulness information, an evaluation that the shorter a recovery time in which the user becomes awake after the end of the nap is, the higher the quality of the nap.

8. The nap evaluation device of claim 7, wherein
the evaluation unit evaluates the quality of the nap further with reference to the wakefulness information at a timing at which the prescribed time elapses since the end of the nap.

9. The nap evaluation device of claim 1, further comprising an output unit configured to output an evaluation result by the evaluation unit.

10. A nap evaluation system, comprising:
the nap evaluation device of claim 1; and
a display configured to present an evaluation result by the evaluation unit.

11. The nap evaluation system of claim 10, wherein
the display is configured to present the evaluation result by the evaluation unit and a past evaluation result acquired from a storage unit configured to store evaluation results by the evaluation unit in a comparative manner.

12. A nap evaluation method, comprising:
a first acquisition process of acquiring time information regarding a time during which a user is napping;
a second acquisition process of acquiring a sleep stage of the user who is napping; and
an evaluation process of evaluating a quality of a nap in accordance with the time information and the sleep stage,
in the evaluation process, an evaluation being made that the lower a ratio of a second sleep time to a first sleep time is, the higher the quality of the nap,
the first sleep time being a sleep time in light sleep during which the sleep stage is relatively light in a nap time from a start to an end of the nap, the second sleep time being a sleep time in deep sleep during which the sleep stage is relatively deep in the nap time.

13. A computer-readable, non-transitory, and tangible recording medium recording a program to be stored in a computer, the program causing the computer to execute the nap evaluation method of the claim 12.

14. The nap evaluation device of claim 2,
the evaluation unit makes an evaluation that the shorter a time of a wakefulness state between a start to an end of the nap is, the higher the quality of the nap.

15. The nap evaluation device of claim 2, wherein
the sleep stage is a value determined with reference to biological information on the user.

16. The nap evaluation device of claim 3, wherein
the sleep stage is a value determined with reference to biological information on the user.

17. The nap evaluation device of claim 2, further comprising a third acquisition unit configured to acquire a sleep onset time which is a time until the user falls asleep, wherein
the evaluation unit further makes an evaluation that the shorter the sleep onset time is, the higher the quality of the nap.

18. The nap evaluation device of claim 3, further comprising a third acquisition unit configured to acquire a sleep onset time which is a time until the user falls asleep, wherein
the evaluation unit further makes an evaluation that the shorter the sleep onset time is, the higher the quality of the nap.

19. The nap evaluation device of claim 4, further comprising a third acquisition unit configured to acquire a sleep onset time which is a time until the user falls asleep, wherein
the evaluation unit further makes an evaluation that the shorter the sleep onset time is, the higher the quality of the nap.

20. The nap evaluation device of claim 2, wherein
the light sleep includes a sleep stage 2 which is second lightest of four sleep stages 1 to 4 into which non-REM sleep is classified from light to deep sleep, and
the evaluation unit further makes an evaluation that the longer a continuation time for which the sleep stage 2 continues is, the higher the quality of the nap.
